(19) United States
(12) Patent Application Publication
(10) Pub. No.: US 2026/0210924 A1
(43) Pub. Date: Jul. 23, 2026

(54) GAS DETECTION SYSTEM

(71) Applicant: GE Vernova Infrastructure Technology LLC, Greenville, SC (US)

(72) Inventors: Rajesh Reddy KONATHAM, Karnataka (IN); Jeevankumar KRISHNAN, Karnataka (IN)

(21) Appl. No.: 19/140,821

(22) PCT Filed: Dec. 18, 2023
(86) PCT No.: PCT/US2023/084538

§ 371 Date: Jun. 18, 2025

(30) Foreign Application Priority Data
Dec. 23, 2022 (IN) 202211075044

(51) Int. Cl. G01N 33/00; F02C 9/00; G08B 21/14

(52) U.S. Cl. CPC G01N 33/0031; F02C 9/00; G01N 33/0006; G01N 33/0063; G08B 21/14

(57) ABSTRACT

A gas detection method and system for detecting a predetermined gas in a system is disclosed. A gas detection system includes a valve system for initially positioning a first gas sensor in fluid communication with a first sensing location and a second gas sensor in fluid communication with a second sensing location. In response to detecting the predetermined gas with a selected sensor from a selected sensing location, the other sensor that did not detect the predetermined gas is placed in fluid communication with the selected sensing location for which the selected sensor sensed the predetermined gas. If the other sensor also detects the predetermined gas in the selected sensing location, an alarm occurs. If the other sensor does not detect the predetermined gas in the selected sensing location, the selected sensor is recalibrated.

### TECHNICAL FIELD

The disclosure relates generally to a gas detection system for use with a gas turbine engine. More particularly, this disclosure relates to a gas detection system including a valve system for switching gas sensors between separate sensing locations for checking accuracy of a gas sensor that senses a hazardous gas (haz gas).

### BACKGROUND

Gas turbines and/or generators (e.g., hydrogen-cooled generators) are used to generate power for various applications. To protect the turbine and/or generator from the surrounding environment and vice versa, the turbine and/or generator may be housed or enclosed in an enclosure with appropriate inlets, exhaust outlets, and/or ventilation systems. For example, a gas turbine and/or generator may be housed inside an enclosure, which may facilitate reducing noise during turbine operation and which may prevent environmental hazards such as combustible gases (e.g., fuel gas or hydrogen) from leaking to the surrounding environment. A monitoring system may be fluidly coupled to an enclosure to sample the air within the enclosure to detect the presence of hazardous gas. Unfortunately, under certain conditions, these monitoring systems may trip the power generation units (e.g., due to moisture or ice in the sensing lines), resulting in unnecessary and costly shutdowns. Due to this issue, certain operators may inactivate monitoring systems and forego monitoring for hazardous gas within the enclosure.

There are many situations in which hazardous gases may accumulate in dangerous concentrations. In such cases, health and safety regulations and prudence both require a system capable of detecting accumulations of hazardous gases before a dangerous situation arises. For example, many industrial processes use highly flammable or poisonous gases. An industrial plant which uses such processes typically requires a gas detection system having gas sensors distributed throughout the plant and a central station which receives signals from the gas sensors. If one of the gas sensors detects an excessive amount of hazardous gas, then an alarm condition is tripped at the central station. Such industrial gas detection systems are typically expensive. The central stations typically include proprietary hardware which has limited upgradability. Gas sensors are available for detecting a wide range of hazardous gases. Sensors are available for detecting flammable gases, asphyxiating gases of various kinds, radioactive gases, gases containing certain toxins, and so on.

Self-contained gas detection systems are also available. A self-contained gas detection system comprises a gas sensor, a battery, a simple control circuit and an audible and/or visible alarm contained in a small housing. An example of such self-contained gas detectors are the carbon monoxide detectors and smoke detectors which are widely marketed for use in households and small businesses.

Industrial plants must typically have both built-in gas detection systems and portable self-contained gas detectors. If the built-in system detects a troubling amount of a hazardous gas in the vicinity of a particular gas sensor, then personnel may be dispatched to the area of the gas sensor in question with portable gas sensors. Portable gas sensors may be used to confirm the amount of hazardous gas detected and to locate the source of the hazardous gas. The management of such industrial plants typically has rigid policies in place which require measurements made by plant personnel to be carefully documented.

### BRIEF DESCRIPTION

All aspects, examples and features mentioned below can be combined in any technically possible way.

An aspect of the disclosure provides a gas detection method for detecting a predetermined gas in a system, the system including a first sensing location and a second sensing location, a valve system including at least two valves for selectively positioning a first gas sensor in fluid communication with at least one of the first sensing location and the second sensing location and a second gas sensor in fluid communication with at least one of the first sensing location and the second sensing location, and a controller controlling the valve system, the method comprising: setting the valve system in a first setting in which the first gas sensor is in fluid communication with only the first sensing location to detect the predetermined gas in only the first sensing location, and the second gas sensor is in fluid communication with only the second sensing location to detect the predetermined gas in only the second sensing location; in response to detecting the predetermined gas with a tripped sensor of the first gas sensor in the first sensing location or the second gas sensor in the second sensing location, switching the valve system to a second setting in which a non-tripped sensor of the first gas sensor or the second gas sensor that did not detect the predetermined gas is in fluid communication with the first sensing location or the second sensing location in which the predetermined gas was detected; in response to the non-tripped sensor detecting the predetermined gas in the first sensing location or the second sensing location in which the predetermined gas was detected by the tripped sensor, initiating an alarm; and in response to the non-tripped sensor not detecting the predetermined gas in the first sensing location or the second sensing location in which the predetermined gas was detected by the tripped sensor, recalibrating the tripped sensor that detected the predetermined gas.

Another aspect of the disclosure includes any of the preceding aspects, and further including confirming calibration of the selected gas sensor that detected the predetermined gas while in operation after the recalibrating.

Another aspect of the disclosure includes any of the preceding aspects, and the first sensing location includes a gas turbine compartment and a gas turbine, and further including the controller enabling continued operation of the gas turbine in response to determining that the selected gas sensor that detected the predetermined gas is operating after the recalibration.

Another aspect of the disclosure includes any of the preceding aspects, and the predetermined gas includes a hazardous gas.

Another aspect of the disclosure includes any of the preceding aspects, and further including directing gas by a source of negative pressure from one of the first sensing location and the second sensing location to each of the first gas sensor and the second gas sensor.

Another aspect of the disclosure includes any of the preceding aspects, and the source of negative pressure includes a fan or an ejector.

Another aspect of the disclosure includes any of the preceding aspects, and the valve system includes at least one solenoid valve.

Another aspect of the disclosure includes any of the preceding aspects, and the valve system includes at least two three-way valves.

Another aspect of the disclosure includes any of the preceding aspects, and the valve system includes at least two two-way valves.

Another aspect of the disclosure includes any of the preceding aspects, and the first sensing location and the second sensing location each include a source of negative pressure, each source of negative pressure being connected to the respective gas sensor.

Another aspect of the disclosure includes any of the preceding aspects, and the first sensing location and the second sensing location are each connected to the respective gas sensor by at least one sampling conduit and the valve system.

An aspect of the disclosure includes a gas detection system for detecting a predetermined gas, the system comprising: a first sensing location and a second sensing location; a first gas sensor and a second gas sensor; a valve system including at least two valves for selectively positioning the first gas sensor in fluid communication with at least one of the first sensing location and the second sensing location and the second gas sensor in fluid communication with at least one of the first sensing location and the second sensing location; and a controller controlling the valve system in response to signals from the first gas sensor and the second gas sensor, the controller enabling: setting the valve system in a first setting in which the first gas sensor is in fluid communication with only the first sensing location to detect the predetermined gas in only the first sensing location and the second gas sensor is in fluid communication with only the second sensing location to detect the predetermined gas in only the second sensing location; in response to detecting the predetermined gas with a tripped sensor of the first gas sensor in the first sensing location or the second gas sensor in the second sensing location, switching the valve system to a second setting in which a non-tripped sensor of the first gas sensor or the second gas sensor that did not detect the predetermined gas is in fluid communication with the first sensing location or the second sensing location in which the predetermined gas was detected; in response to the non-tripped sensor detecting the predetermined gas in the first sensing location or the second sensing location in which the predetermined gas was detected by the tripped sensor, initiating an alarm; and in response to the non-tripped sensor not detecting the predetermined gas in the first sensing location or the second sensing location in which the predetermined gas was detected by the tripped sensor, recalibrating the tripped sensor that detected the predetermined gas.

Another aspect of the disclosure includes any of the preceding aspects, and the controller enables confirming calibration of the selected sensor that detected the predetermined gas while in operation after recalibration.

Another aspect of the disclosure includes any of the preceding aspects, and the first sensing location includes a gas turbine compartment and a gas turbine, wherein the controller enables continued operation of the gas turbine in response to determining that the selected sensor is operating after the recalibrating.

Another aspect of the disclosure includes any of the preceding aspects, and the predetermined gas includes a hazardous gas.

Another aspect of the disclosure includes any of the preceding aspects, and further comprising a source of negative pressure connected to each of the first sensing location and the second sensing location to direct gas to each of the first gas sensor and the second gas sensor.

Another aspect of the disclosure includes any of the preceding aspects, and the source of negative pressure includes a fan.

Another aspect of the disclosure includes any of the preceding aspects, and the valve system includes at least two solenoid three-way valves.

Another aspect of the disclosure includes any of the preceding aspects, and the valve system includes at least two solenoid two-way valves.

Another aspect of the disclosure includes any of the preceding aspects, and the first sensing location and the second sensing location are each connected to the respective gas sensor by at least one sampling conduit and the valve system.

Two or more aspects described in this disclosure, including those described in this summary section, may be combined to form implementations not specifically described herein.

The details of one or more implementations are set forth in the accompanying drawings and the description below. Other features, objects and advantages will be apparent from the description and drawings, and from the claims.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 illustrates a block diagram of a conventional gas detection system;

FIG. 2 illustrates a block diagram of a gas detection system, according to embodiments of the disclosure;

FIGS. 3A, 3B and 3C illustrate block diagrams of settings of a gas detection system, according to embodiments of the disclosure;

FIGS. 4A, 4B and 4C illustrate block diagrams of settings of a gas detection system, according to further embodiments of the disclosure;

FIG. 5 is a flow diagram of a method of gas detection, according to embodiments of the disclosure;

FIGS. 6A and 6B illustrate block diagrams of settings of a gas detection system, according to other embodiments of the disclosure; and

FIGS. 7A and 7B illustrate block diagrams of settings of a gas detection system, according to other embodiments of the disclosure.

It is noted that the drawings of the disclosure are not necessarily to scale. The drawings are intended to depict only typical aspects of the disclosure and therefore should not be considered as limiting the scope of the disclosure. In the drawings, like numbering represents like elements between the drawings.

### DETAILED DESCRIPTION

As an initial matter, in order to clearly describe the subject matter of the current disclosure, it will become necessary to select certain terminology when referring to and describing relevant machine components within hazardous gas detection systems. To the extent possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

In addition, several descriptive terms may be used regularly herein, and it should prove helpful to define these terms at the onset of this section. These terms and their definitions, unless stated otherwise, are as follows. As used herein, “downstream” and “upstream” are terms that indicate a direction relative to the flow of a fluid, such as the flow of air or gas through conduits or systems, including but not limited to hazardous gas detection systems. The term “downstream” corresponds to the direction of flow of the fluid, and the term “upstream” refers to the direction opposite to the flow (i.e., the direction from which the flow originates). The terms “forward” and “aft,” without any further specificity, refer to directions, with “forward” referring to the front end of a hazardous gas detection system, and “aft” referring to a rearward section of the hazardous gas detection system.

In addition, several descriptive terms may be used regularly herein, as described below. The terms “first,” “second,” and “third” may be used interchangeably to distinguish one component from another and are not intended to signify location or importance of the individual components.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components but do not preclude the presence or addition of one or more other features, integers, steps, operations, elements, components, and/or groups thereof. “Optional” or “optionally” means that the subsequently described event or circumstance may or may not occur or that the subsequently described component or element may or may not be present, and that the description includes instances where the event occurs or the component is present and instances where it does not or is not present.

Where an element or layer is referred to as being “on,” “engaged to,” “connected to” or “coupled to” another element or layer, it may be directly on, engaged to, connected to, or coupled to the other element or layer, or intervening elements or layers may be present. In contrast, when an element is referred to as being “directly on,” “directly engaged to,” “directly connected to” or “directly coupled to” another element or layer, no intervening elements or layers are present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

A gas detection method and system for detecting a predetermined gas in a system is described herein. In certain embodiments, a gas detection system includes a valve system for initially positioning a first gas sensor in fluid communication to a first sensing location and a second gas sensor in fluid communication with a second sensing location. In response to detecting the predetermined gas with a tripped sensor from one of the sensing locations, the other, non-tripped sensor that did not detect the predetermined gas is placed in fluid communication with the sensing location for which the tripped sensor sensed the predetermined gas. If the non-tripped sensor also detects the predetermined gas in the sensing location, an alarm occurs. If the non-tripped sensor does not detect the predetermined gas in the selected sensing location, the initially tripped sensor is recalibrated. The gas detection system is intended for monitoring presence of hazardous gases (e.g., combustible gases, such as a fuel gas (e.g., natural gas) in a gas turbine enclosure or hydrogen in a generator enclosure) inside an equipment area (e.g., an enclosure) for a turbomachine (e.g., a gas turbine, a generator, etc.). The gas detection system includes a controller with freezing and moisture buildup protection. The system includes multiple sampling conduits that provide gas samples from different sensing locations (where sample probes are located) in two or more enclosures. Each sampling conduit may include a flow monitor configured to proactively monitor and determine if the sample flow drops to an unacceptable level.

In other embodiments, the gas detection system may include a pair of gas sensors coupled to a single sensing location with two sources of negative pressure (perhaps in a single gas detection controller) coupled thereto.

Aspects of the disclosure thus provide a system and method for a gas detection system that monitors and detects (also referred to as “senses”) gas and also reduces number of gas detection controllers and/or gas sensors required. As embodied by the disclosure, the system and method include a valve system including at least two valves, for example, two-way or three-way valves. The two-way or three-way valves may be solenoid valves. In certain embodiments, the system can sample from two different sensing locations through various sampling conduits. Hence, the system and method may include only one controller for numerous sensing locations (e.g., independent ventilation sampling conduits, two different compartments, etc.) to determine if the gas includes a predetermined gas, such as but not limited to a hazardous gas. As noted, in other embodiments, the gas detection system may include a pair of gas sensors coupled to a single sensing location with two sources of negative pressure (perhaps in a single gas detection controller) coupled thereto.

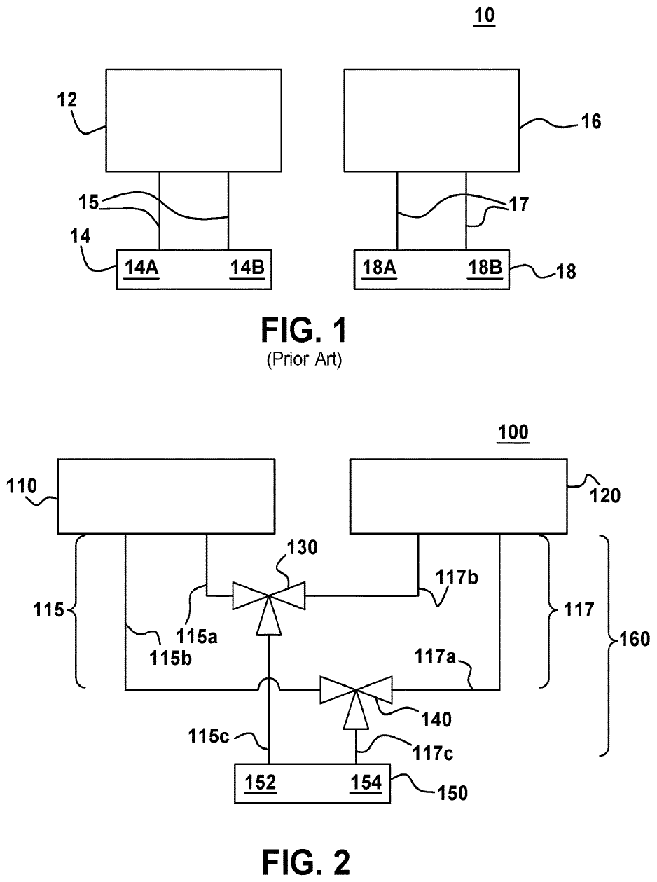

Current systems and methods for sampling gas have dedicated sampling tubing and gas detection controllers for separate sensing locations. As in FIG. 1, the current gas detection system 10 includes two gas detection controllers 14 and 18 for two compartments 12 and 16, respectively. Current gas detection system 10 includes independent sampling conduits 15 and 17, respectfully, attached to each of the compartments 12 and 16 and leading to gas detection controllers 14 and 18. Further, a source of pressure (not illustrated for ease of understanding), such as fans, can be located in compartments 12 and 16 to draw or move gas from compartments 12 and 16 to gas detection controllers 14 and 18. The source of pressure may be viewed as a negative source of pressure directing gas from compartments 12 and 16 to gas detection controllers 14 and 16. Gas detection controllers 14 and 16 include sensors 14A and 14B and sensors 18A and 18B respectively to detect the predetermined gas. Moreover, gas detection controllers 14 and 18 include controls to provide signals if the predetermined gas is detected. Signals can be used to alert operators or monitors to potential hazardous gas situations. In a similar situation to FIG. 1, where a single sensing location includes two sources of negative pressure for different exhaust ducts, two gas detection controllers are used, one for each source of negative pressure. In any event, multiple components doing similar functions may increase the cost of current gas detection system 10 and increase maintenance of current gas detection system 10.

FIGS. 2-4C illustrate embodiments of a gas detection system for detecting a predetermined gas in a system (e.g., a gas turbine system) including two or more sensing locations, according to the disclosure. The different embodiments include variations of a valving system 160 that may include at least two valves. For example, FIG. 2 illustrates a gas detection system 100 including a valve system 160 including two three-way solenoid valves 130, 140. As embodied by the disclosure, valve system 160 can include all solenoid valves and/or a variety of manual valves. For purposes of description, dark or black shading on the valve portion indicates it is closed to flow, and light or white shading on the valve portion indicates it is open to flow.

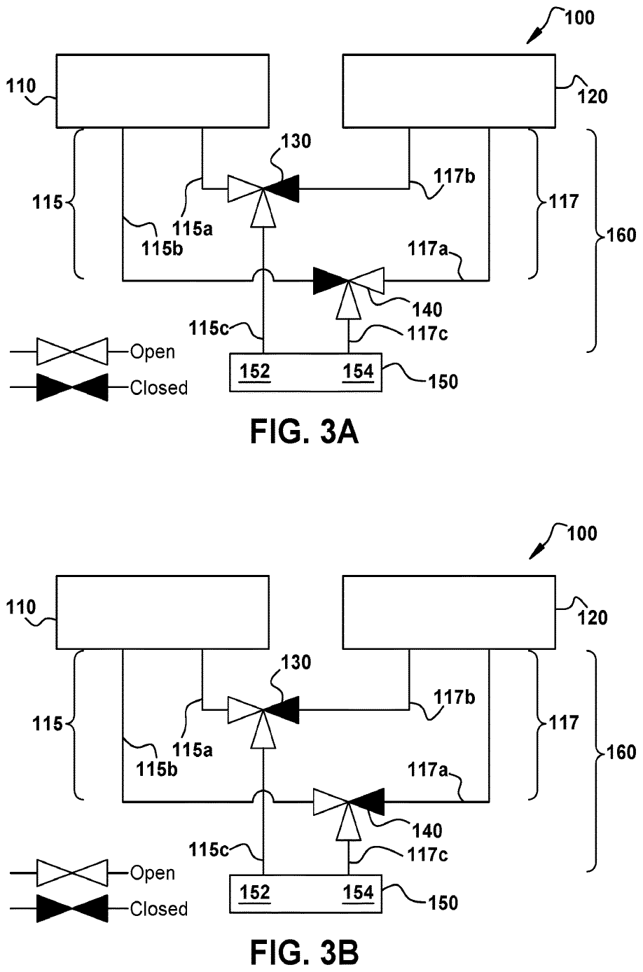

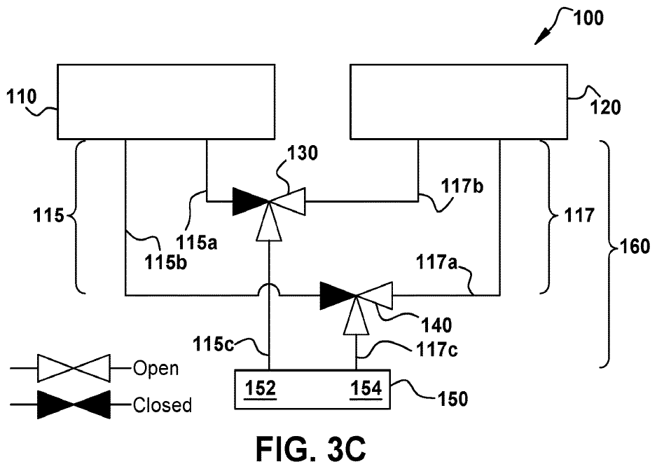

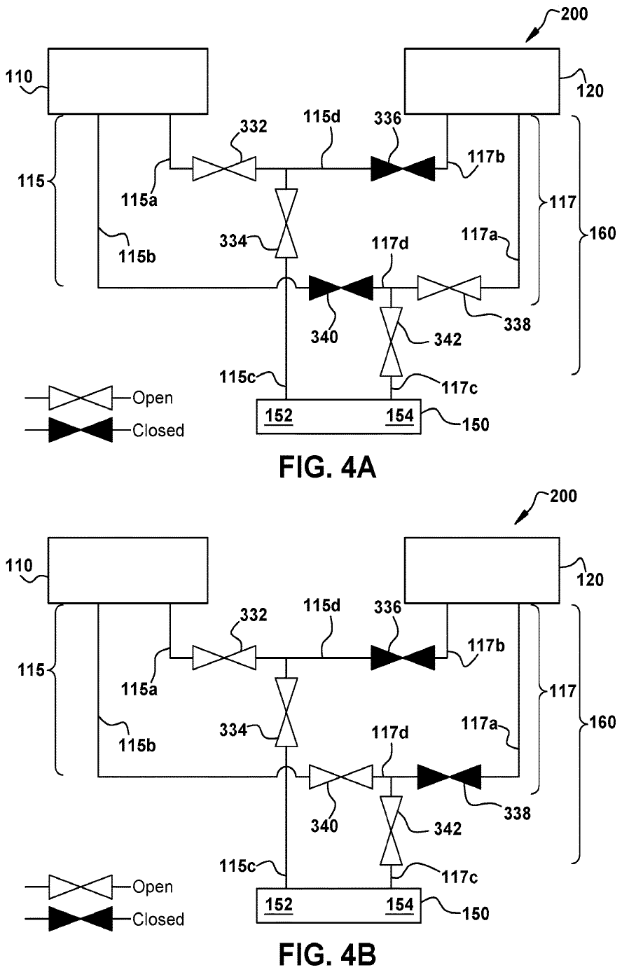

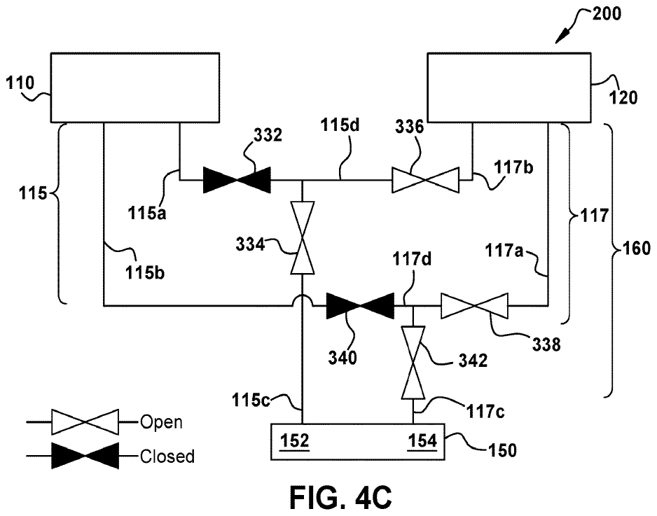

FIG. 2 illustrates gas detection system 100 (hereafter “system 100”) with valve system 160 including at least two three-way valves 130 and 140. FIGS. 3A-C illustrate settings of system 100 of FIG. 2 during the operation of system 100 according to embodiments of the disclosure. As will be further described herein, FIGS. 4A-B illustrate a gas detection system 200 (hereafter “system 200”) with valve system 160 including at least six two-way valves 332, 334, 336, 338, 340, 342.

With respect to FIG. 2, system 100 includes valve system 160 including at least two valves 130, 140 for selectively positioning a first gas sensor 152 in fluid communication with at least one of a first sensing location 110 and a second sensing location 120, and a second gas sensor 154 in fluid communication with at least one of first sensing location 110 and second sensing location 120.

System 100 also includes a controller 150, also referred to as a gas detection controller. Controller 150 controls valve system 160, among other things. That is, controller 150 includes controls for moving valves of valve system 160, as described herein. Controller 150 may also optionally include first and second gas sensors 152, 154 for detecting if a predetermined gas is present in an apparatus associated with system 100. Alternatively, gas sensors 152, 154 can be separate from, but operatively coupled to, controller 150. In any event, gas sensors 152 and 154 analyze the input gas drawn from sensing locations 110, 120 of the apparatus, described herein. Also, controller 150 includes logic that can provide signals to valves of valve system 160 for opening and closing individual valves of valve system 160, as embodied by the disclosure and described herein.

System 100 may include or be operatively coupled to first sensing location 110 and second sending location 120. In accordance with certain aspects of the embodiments, first sensing location 110 is a gas turbine compartment with a gas turbine therein, the gas turbine being part of a gas turbine system. Second sensing location 120 is a gas module of the gas turbine system, which supplies fuel to the gas turbine. In other embodiments, first sensing location 110 may be in a first area of a gas turbine compartment or a gas module, and second sensing location may be in a different, second area of the same gas turbine compartment or gas module. It will be recognized that the sensing locations 110, 120 can be any area in which gas detection is desired.

System 100 may also include independent sampling conduit systems 115 and 117 coupling first gas sensor 152 and/or second gas sensor 154 to first and/or second sensing locations 110, 120. Sampling conduit system 115 includes a sampling conduit 115a that fluidly connects first sensing location 110 to three-way valve 130 of valve system 160. Sampling conduit system 115 includes a sampling conduit 115b that fluidly connects first sensing location 110 to three-way valve 140 of valve system 160. Additionally, sampling conduit system 115 includes a sampling conduit 115c that fluidly connects three-way valve 130 of valve system 160 to first gas sensor 152.

Sampling conduit system 117 includes a sampling conduit 117a that fluidly connects second sensing location 120 to three-way valve 140 of valve system 160. Sampling conduit system 117 includes a sampling conduit 117b that fluidly connects second sensing location 120 to three-way valve 130 of valve system 160. Sampling conduit system 117 includes a sampling conduit 117c that fluidly connects three-way valve 140 of valve system 160 to second gas sensor 154.

A source of pressure (not illustrated for ease of understanding), such as one or more fans, can be located with first gas sensor 152 or second gas sensor 154 to draw or move gas from first sensing location 110 and second sensing location 120 to gas sensors 152, 154. Alternatively, a source of negative pressure (not illustrated for ease of understanding), such as one or more fans, can be located in first sensing location 110 and second sensing location 120 to draw or move gas from first sensing location 110 and second sensing location 120 to gas sensors 152, 154. Thus, the source of negative pressure may direct gas from first sensing location 110 and second sensing location 120 to gas sensors 152, 154.

As embodied by the disclosure, system 100 includes a controller 150. Controller 150 may include or be operatively coupled to a pair of gas sensors 152, 154 respectively to detect the predetermined gas. Fluid communication between first sensing location 110 and gas sensors 152, 154 can occur via first sampling conduit system 115 and valve system 160, and fluid communication between second sensing location 120 and gas sensors 154, 152 can occur via second sampling conduit system 117 and valve system 160. Moreover, controller 150 includes an interface to provide signals if the predetermined gas is detected. Signals can be used to alert operators to potential hazardous gas situations or to shut down a related apparatus such as a gas turbine, as discussed hereinafter.

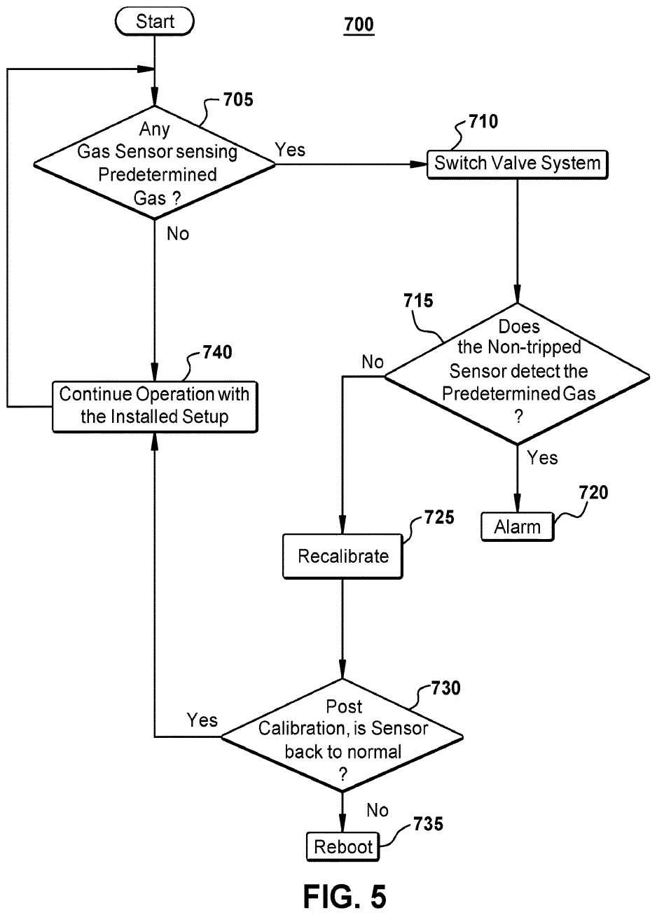

In accordance with the operation of gas detection system 100, as illustrated in FIG. 3A and the flow diagram of FIG. 5, embodiments of the disclosure provide a method 700 to detect gas. In a first setting of valve system 160, first gas sensor 152 is in fluid communication with only first sensing location 110 to detect the predetermined gas in only first sensing location 110. That is, first sensing location 110 is fluidly connected to first gas sensor 152 through three-way valve 130 of valve system 160. More particularly, first sensing location 110 is fluidly connected to gas sensor 152 through three-way valve 130 of valve system 160 via sampling conduit 115a, three-way valve 130, and sampling conduit 115c. Further, as seen in FIG. 3A, in this first setting of valve system 160, second gas sensor 154 is in fluid communication with only second sensing location 120 to detect the predetermined gas in only second sensing location 120. That is, second sensing location 120 is fluidly connected to second gas sensor 154 through three-way valve 140 of valve system 160. More particularly, second sensing location 120 is connected to gas sensor 154 through three-way valve 140 of valve system 160 via sampling conduit 117a, three-way valve 140, and sampling conduit 117c.

In process 705 in FIG. 5, gas sensors 152, 154 monitor for the presence of the predetermined gas in the respective sensing locations 110, 120. A gas sensor 152, 154 that initially detects the predetermined gas is referred to herein as a “tripped sensor” because it is activated or tripped, i.e., it has detected the presence of the predetermined gas. A gas sensor 152, 154 that does not initially detect the predetermined gas is referred to herein as a “non-tripped sensor” because it is not initially activated or tripped, i.e., it has not detected the gas.

In response to detecting the predetermined gas with a tripped sensor of first gas sensor 152 in first sensing location 110 or second gas sensor 154 in second sensing location 120, i.e., Yes at process 705, controller switches valve system 160 at process 710. More particularly, as shown in FIGS. 3B and 3C, controller 150 switches valve system 160 (at process 710) to a second setting in which a ‘non-tripped sensor’ of first gas sensor 152 or second gas sensor 154 that did not detect the predetermined gas is in fluid communication with first sensing location 110 or second sensing location 120 in which the predetermined gas was detected. In other words, the other sensor of first gas sensor 152 or second gas sensor 154, i.e., the non-tripped sensor, which did not detect the predetermined gas from fluid communication with first sensing location 110 or second sensing location 120 is placed in fluid communication with the first sensing location 110 or second sensing location 120 in which gas was detected. In the example in FIG. 3B, gas was detected by first gas sensor 152 in first sensing location 110, and controller 150 has switched three-way valve 140 to fluidly connect second gas sensor 154, i.e., the non-tripped sensor, and first sensing location 110 in which gas was detected by first gas sensor 152. More particularly, first sensor location 110 is fluidly connected to second gas sensor 154 by sampling conduit 115b, three-way valve 140 and sampling conduit 117c. In this configuration, both first gas sensor 152 and second gas sensor 154 are in fluid communication with first sensing location 110.

Conversely, as shown in FIG. 3C, if a predetermined gas is initially detected at second sensing location 120 by second gas sensor 154, i.e., the tripped sensor, controller 150 will cause valve system 160 to switch (i.e., using three-way valve 130) to a second setting in which second sensing location 120 is fluidly connected to first gas sensor 152, i.e., through three-way valve 130 via sampling conduit 117b, three-way valve 130, and sampling conduit 115c. In this configuration, both first gas sensor 152 and second gas sensor 154 are in fluid communication with second sensing location 120.

In other words, detection of the predetermined gas by a first, tripped gas sensor from one of either first sensing location 110 or second sensing location 120 will cause switching of the valve(s) of valve system 160 corresponding to the non-tripped sensor, e.g., the respective three-way valves 130, 140, to fluidly connect the sensing location in which gas was detected to the other, non-tripped sensor.

The switching enables the non-tripped gas sensor (154 in FIGS. 3B, 152 in FIG. 3C) to confirm detection of the predetermined gas. At process 715, in response to the other, non-tripped sensor detecting the predetermined gas in first sensing location 110 or second sensing location 120 in which the predetermined gas was detected by the tripped sensor (152 in FIGS. 3B, 154 in FIG. 3C), i.e., Yes at process 715, controller 150 initiates an alarm at process 720. That is, in accordance with the operational methodology of system 100 as embodied by the disclosure, where controller 150 confirms detection of the predetermined gas, it initiates an alarm, which can lead to any variety of operator-initiated or system-initiated remedial action, e.g., additional operator investigation, shutdown of the gas turbine, etc.

Alternatively, in response to the non-tripped sensor (154 in FIGS. 3B, 152 in FIG. 3C) not detecting the predetermined gas in first sensing location 110 or second sensing location 120 in which the predetermined gas was detected by the tripped sensor (152 in FIGS. 3B, 154 in FIG. 3C), i.e., No at process 715, controller 150 recalibrates the tripped gas sensor that detected the predetermined gas at process 725. Controller 150 can also generate an alarm to notify an operator of the recalibration occurring. That is, if no confirmation of detecting the predetermined gas after switching occurs, a recalibration of the tripped sensor that initially detected the predetermined gas can be undertaken.

FIGS. 4A-C illustrate a further embodiment of a gas detection system 200 (hereafter “system 200”) according to embodiments of the disclosure. In system 200, valve system 160 includes a set of two-way valves 332, 334, 336, 338, 340, and 342. Two-way valves 332, 334, 336, 338, 340, and 342 are connected to first sensing location 110 and second sensing location 120 by sampling conduit systems 115, 117 including sampling conduits 115a, 115b, 115c, 115d, and 117a, 117b, 117c, and 117d. As embodied by the disclosure, opening and closing of two-way valves 332, 334, 336, 338, 340, and 342 by controller 150 will provide similar communication of gas from first sensing location 110 and second sensing location 120 to first and second sensors 152 and 154 as in the FIGS. 3A-C embodiment with three-way valves 130 and 140. Additionally, as noted herein, the source of negative pressure for system 200, as embodied by the disclosure, can include a fan or an ejector to pull gas from first sensing location 110 and second sensing location 120, e.g., in the locations and/or as part of gas sensor 152, 154 and/or controller 150.

In accordance with the operation of system 200, as illustrated in FIG. 4A, in a first setting, first sensing location 110 is connected to first gas sensor 152 through two-way valves 332 and 334 of valve system 160. More particularly, in the first setting of system 200, first sensing location 110 is connected to first gas sensor 152 through two-way valves 332 and 334 via sampling conduit 115a, two-way valve 332, sampling conduit 115d, two-way valve 334, and sampling conduit 115c. In the first setting of system 200, second sensing location 120 is connected to second gas sensor 154 through two-way valves 338 and 342 of valve system 160 via sampling conduit 117a, two-way valve 338, sampling conduit 117d, two-way valve 342, and sampling conduit 117c. Moreover, in the first setting of system 200, two-way valves 332, 334, 338, 342 are open and two-way valves 336 and 340 are closed.

FIGS. 4B and 4C show two different possibilities of the second setting based on which sensing location detects the predetermined gas.

As shown in FIG. 4B, and with reference to the flow diagram of FIG. 5, if a predetermined gas is detected at first sensing location 110 (Yes at step 705) by first gas sensor 152, i.e., the tripped gas sensor, controller 150 will switch valve system 160 by closing two-way valve 338 and opening two-way valve 340 at process 710. Thus, when predetermined gas is detected from first sensing location 110 by first gas sensor 152, first sensing location 110 is fluidly connected to second gas sensor 154, i.e., the non-tripped sensor, through two-way valve 340 and two-way valve 342. More particularly, the fluid connection of first sensing location 110 to second gas sensor 154 is via sampling conduit 115b, two-way valve 340, sampling conduit 117d, two-way valve 342, and sampling conduit 117c. Also, first sensing location 110 remains connected to first gas sensor 152 through two-way valves 332 and 334 via sampling conduit 115a, two-way valve 332, sampling conduit 115d, two-way valve 334, and sampling conduit 115c.

Conversely, as shown in FIG. 4C, and with reference to FIG. 5, if a predetermined gas is detected from second sensing location 120 (Yes at process 705) by second gas sensor 154, i.e., the tripped gas sensor, controller 150 will switch valve system 160 to cause two-way valve 336 to switch from closed to open and cause two-way valve 332 to switch from open to closed at process 710. In the switched position of two-way valve 336, second sensing location 120 is connected to first gas sensor 152, i.e., the non-tripped sensor, through two-way valve 336 and two-way valve 334 of valve system 160 via sampling conduit 117b, two-way valve 336, sampling conduit 115d, two-way valve 334, and sampling conduit 115c. Further, second sensing location 120 remains connected to second gas sensor 154 through two-way valves 338 and 342 of valve system 160 via sampling conduit 117a, two-way valve 338, sampling conduit 117d, two-way valve 342, and sampling conduit 117c.

The switching will enable the non-tripped gas sensor (154 in FIGS. 4B, 152 in FIG. 4C) to confirm detection of the predetermined gas. More particularly, in response to the non-tripped sensor detecting the predetermined gas in first sensing location 110 or second sensing location 120 in which the predetermined gas was detected (Yes at process 705) by the tripped sensor (152 in FIGS. 4B, 154 in FIG. 4C), controller 150 initiates an alarm at process 720. That is, in accordance with the operational methodology of system 200 as embodied by the disclosure, where controller 150 confirms detection of the predetermined gas, it initiates an alarm, which can lead to any variety of operator-initiated or system-initiated remedial action, e.g., additional operator investigation, shutdown of the gas turbine, etc. Alternatively, in response to the non-tripped sensor not detecting the predetermined gas in first sensing location 110 or second sensing location 120 in which the predetermined gas was detected by the tripped sensor, i.e., No at process 715, controller 150 recalibrates the tripped gas sensor (152 in FIGS. 4B, 154 in FIG. 4C) that detected the predetermined gas at process 725. That is, if no confirmation of detection of the predetermined gas after switching occurs, a recalibration of the gas sensor that initially detected the predetermined gas can be undertaken. Operators can also be notified of the recalibration.

With further reference to FIG. 5, the recalibration at process 725 occurs when the tripped gas sensor 152 or 154 that initially detected the predetermined gas from its respective first sensing location 110 and second sensing location 120 in step 705 may be faulty, i.e., because the originally non-tripped sensor does not confirm the detection of gas. Thus, the tripped sensor that initially detected the predetermined gas can be checked for proper operation and/or sensitivity and is recalibrated in step 725. Regardless of embodiment of system 100 or 200, once recalibration in step 725 is complete, controller 150, at process 730, monitors the recalibrated gas sensor to confirm a proper calibration while in operation after recalibration. If the operation of the gas sensor is determined still to be faulty (No at process 730), valve system 160 can switch valves of the gas detection system 100 or 200 to attempt a hard “reboot” of that sensor that initially detected the predetermined gas at process 735. If operation of the sensor is determined to be correct or normal, i.e., Yes at process 730, operation of the apparatus utilizing system 100 or 200 continues at process 740. Monitoring with the gas detection system, as embodied by the disclosure, continues at process 705.

Referring again to process 705, if gas sensors 152 and 154 do not detect the predetermined gas from their respective first sensing location 110 and second sensing location 120 (No at process 705), operation of the apparatus utilizing the gas detection system 100 or 200 continues in process 740. That is, monitoring with the gas detection system, as embodied by the disclosure, continues.

While two sensing locations 110, 120 have been disclosed herein, it will be recognized that the teachings of the disclosure can be expanded to operate with any number of sensing locations.

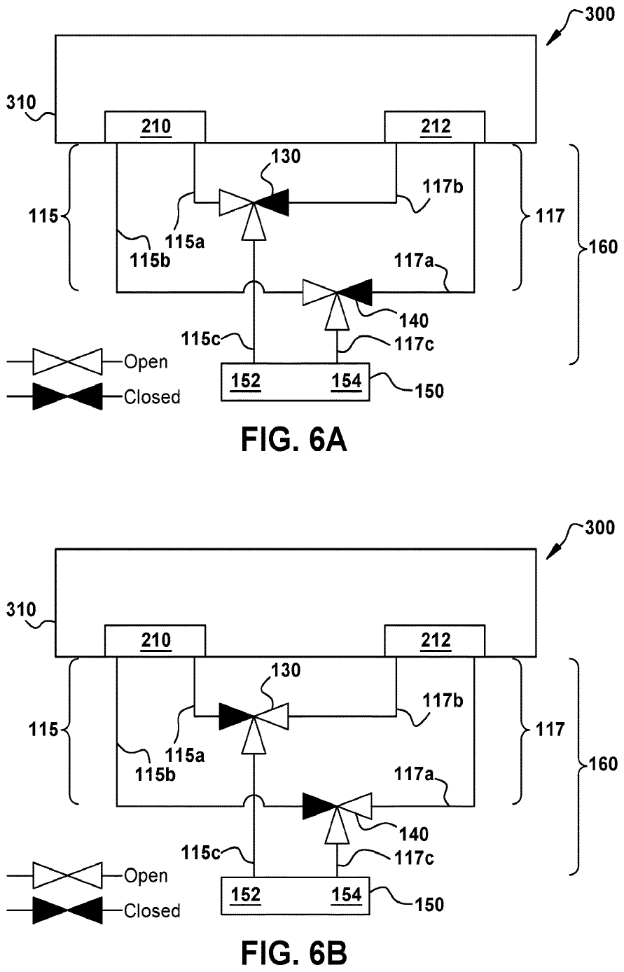

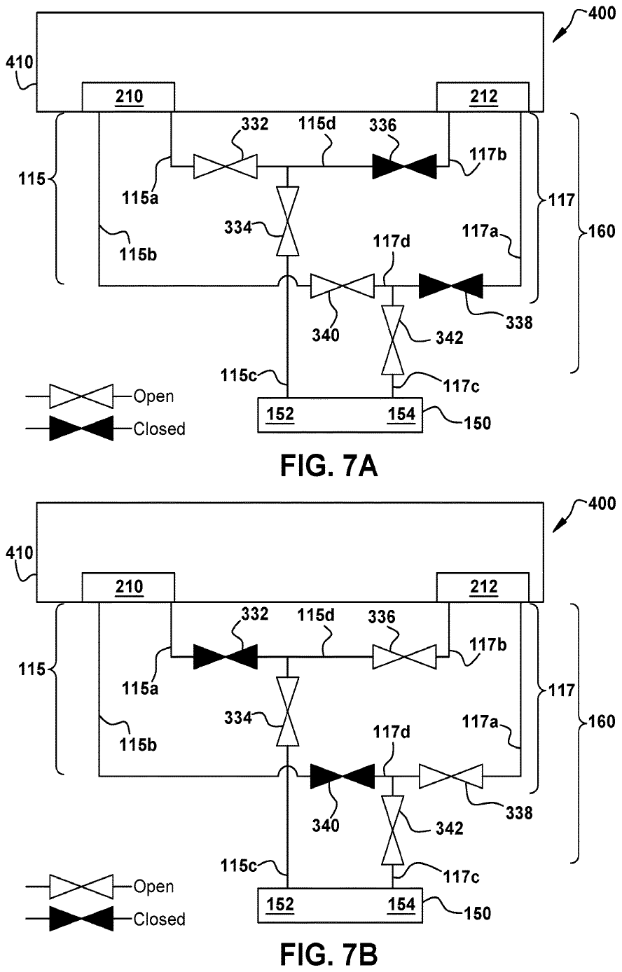

FIGS. 6A-B and 7A-B illustrate further embodiments of a gas detection system 300 or 400 with a reduced number of gas sensors and/or gas detection controllers, respectively, according to other embodiments of the disclosure. FIGS. 6A and 6B have a similar valve system 160 to FIGS. 3B and 3C but only a single sensing location 310 is present, and sensing location 310 includes two sources of negative pressure 210, 212. FIGS. 7A and 7B have a similar valve system 160 to FIGS. 4B and 4C but only a single sensing location 410 is present, and sensing location 410 includes two sources of negative pressure 210, 212.

FIGS. 6A-B and FIGS. 7A-B depict positioning of their valve system 160 dependent on which source of negative pressure 210, 212 (hereafter “source 210” or “source 212” for brevity) is on. As noted herein, the sources of negative pressure for system 300 or 400, as embodied by the disclosure, can include a fan to pull gas from sensing location 310 or 410. While a fan can be employed to be a source of negative pressure to pull gas from sensing location 310 or 410, aspects of the disclosure include other sources of negative pressure. Sources of negative pressure include, but are not limited to: vacuum pumps, venturi systems, displacement pumps, and the like, including other devices that create negative pressure now known or hereinafter developed. Each source of negative pressure 210, 212 may represent a different exhaust path for gas from sensing location 310 or 410.

In FIG. 6A, gas is directed from sensing location 310 and source 210 through sampling conduit 115a to three-way valve 130 of valve system 160 to sampling conduit 115c to first gas sensor 152. Further, gas is directed from sensing location 310 and source 210 through sampling conduit 115b to three-way valve 140 to sampling conduit 117c to second gas sensor 154. Hence, flow from sensing location 310 and source 210 is directed to both gas sensors 152, 154. Flow from sensing location 310 using source 212 does not occur in this setting of gas detection system 300, i.e., valve system 160 prevents it, and source 212 is off.

In FIG. 6B, flow is directed from sensing location 310 by source 212 to gas sensors 152 and 154. As illustrated, the switch in settings directs gas from sensing location 310 using source 212 through sampling conduit 117b to three-way valve 130 of valve system 160 to sampling conduit 115c to first gas sensor 152. Further, gas is directed from sensing location 310 by source 212 through sampling conduit 117a to three-way valve 140 of valve system 160 to sampling conduit 117c to second gas sensor 154. Hence, flow from sensing location 310 and source 212 is directed to both gas sensors 152, 154. Flow from sensing location 310 using source 210 does not occur in this setting of gas detection system 300, i.e., valve system 160 prevents it, and source 210 is off.

As illustrated in FIG. 7A, gas is directed from sensing location 410 by source 210 through sampling conduit 115a to two-way valve 332 to sampling conduit 115d to two-way valve 334 to sampling conduit 115c and to first gas sensor 152. Further, gas is directed from sensing location 410 by source 210 through sampling conduit 115b to two-way valve 340 to sampling conduit 117d to two-way valve 342 to sampling conduit 117c and to second gas sensor 154. Flow from sensing location 410 using source 212 does not occur in this setting of system 400, i.e., negative source of pressure 212 is off.

In FIG. 7B, source 210 in sensing location 410 is not in operation, and source 212 is in operation. In this setting, gas is directed from sensing location 410 by source 212 to first and second gas sensors 152 and 154. As illustrated, gas is directed from location 410 by source 212 through sampling conduit 117b to two-way valve 336 to sampling conduit 115d to two-way valve 334 to sampling conduit 115c and then to first gas sensor 152. Further, gas is directed from sensing location 410 using source 212 through sampling conduit 117a to two-way valve 338 to sampling conduit 117d to two-way valve 342 to sampling conduit 117c and then to second gas sensor 154. Flow from sensing location 410 using source 210 does not occur in this setting of system 400, i.e., source 210 is off.

In operation, as shown in FIGS. 6A and 7A, in a first setting, flow from sensing location 310 or 410 goes to first gas sensor 152 and second sensor 154 using source 210. Source 212 is off, so flow from sensing location 310 or 410 using source 212 does not occur. In FIGS. 6B and 7B, in a second setting, flow from sensing location 310 or 410 goes to first gas sensor 152 and second sensor 154 using source 212. Source 210 is off, so flow from sensing location 310 or 410 using source 210 does not occur. Consequently, in these embodiments, sensors 152, 154 monitor whichever source 210 or 212 is providing a flow from sensing location 310 or 410. In operation, if one of gas sensors 152, 154 detects gas, i.e., a tripped sensor, an alarm is generated indicating the detection of gas, and any appropriate remedial action can be taken, e.g., additional operator investigation. In contrast, when both gas sensors 152, 154 detect gas, i.e., both sensors trip, and the gas turbine is shutdown. Another remedial action could also occur under these circumstances, e.g., an alarm can also sound.

While one sensing location 310, 410 has been disclosed herein, it will be recognized that the teachings of the disclosure can be expanded to operate with any number of sensing locations.

The foregoing drawings show some of the processing associated with several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise. “Approximately,” as applied to a particular value of a range, applies to both end values and, unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiments were chosen and described in order to best explain the principles of the disclosure and their practical application and to enable others of ordinary skill in the art to understand the disclosure and the possibility of various modifications as are suited to the particular uses contemplated.

We claim:

1. A gas detection method for detecting a predetermined gas in a system, the system including a first sensing location and a second sensing location, a valve system including at least two valves for selectively positioning a first gas sensor in fluid communication with at least one of the first sensing location and the second sensing location and a second gas sensor in fluid communication with at least one of the first sensing location and the second sensing location, and a controller controlling the valve system, the method comprising:
setting the valve system in a first setting in which the first gas sensor is in fluid communication with the first sensing location to detect the predetermined gas in the first sensing location, and the second gas sensor is in fluid communication with only the second sensing location to detect the predetermined gas in only the second sensing location;
in response to detecting the predetermined gas with a tripped sensor of the first gas sensor in the first sensing location or the second gas sensor in the second sensing location, switching the valve system to a second setting in which a non-tripped sensor of the first gas sensor or the second gas sensor that did not detect the predetermined gas is in fluid communication with the first sensing location or the second sensing location in which the predetermined gas was detected;
in response to the non-tripped sensor detecting the predetermined gas in the first sensing location or the second sensing location in which the predetermined gas was detected by the tripped sensor, initiating an alarm; and
in response to the non-tripped sensor not detecting the predetermined gas in the first sensing location or the second sensing location in which the predetermined gas was detected by the tripped sensor, recalibrating the tripped sensor that detected the predetermined gas.

2. The method according to claim 1, further including confirming calibration of the selected gas sensor that detected the predetermined gas while in operation after the recalibrating.

3. The method according to claim 2, wherein the first sensing location includes a gas turbine compartment and a gas turbine, and further including the controller enabling continued operation of the gas turbine in response to determining that the selected gas sensor that detected the predetermined gas is operating after the recalibration.

4. The method according to claim 1, wherein the predetermined gas includes a hazardous gas.

5. The method according to claim 1, further including directing gas by a source of negative pressure from one of the first sensing location and the second sensing location to each of the first gas sensor and the second gas sensor.

6. The method according to claim 5, wherein the source of negative pressure includes a fan or an ejector.

7. The method according to claim 1, wherein the valve system includes at least one solenoid valve.

8. The method according to claim 1, wherein the valve system includes at least two three-way valves.

9. The method according to claim 1, wherein the valve system includes at least two two-way valves.

10. The method according to claim 1, wherein the first sensing location and the second sensing location each include a source of negative pressure, each source of negative pressure being connected to the respective gas sensor.

11. The method according to claim 1, wherein the first sensing location and the second sensing location are each connected to the respective gas sensor by at least one sampling conduit and the valve system.

12. A gas detection system for detecting a predetermined gas, the system comprising:
a first sensing location and a second sensing location;
a first gas sensor and a second gas sensor;
a valve system including at least two valves for selectively positioning the first gas sensor in fluid communication with at least one of the first sensing location and the second sensing location and the second gas sensor in fluid communication with at least one of the first sensing location and the second sensing location; and
a controller controlling the valve system in response to signals from the first gas sensor and the second gas sensor, the controller enabling:
setting the valve system in a first setting in which the first gas sensor is in fluid communication with only the first sensing location to detect the predetermined gas in only the first sensing location, and the second gas sensor is in fluid communication with only the second sensing location to detect the predetermined gas in only the second sensing location;
in response to detecting the predetermined gas with a tripped sensor of the first gas sensor in the first sensing location or the second gas sensor in the second sensing location, switching the valve system to a second setting in which a non-tripped sensor of the first gas sensor or the second gas sensor that did not detect the predetermined gas is in fluid communication with the first sensing location or the second sensing location in which the predetermined gas was detected;
in response to the non-tripped sensor detecting the predetermined gas in the first sensing location or the second sensing location in which the predetermined gas was detected by the tripped sensor, initiating an alarm;
and
in response to the non-tripped sensor not detecting the predetermined gas in the first sensing location or the second sensing location in which the predetermined gas was detected by the tripped sensor, recalibrating the tripped sensor that detected the predetermined gas.

13. The gas detection system of claim 12, wherein the controller enables confirming calibration of the selected sensor that detected the predetermined gas while in operation after recalibration.

14. The gas detection system of claim 12, wherein the first sensing location includes a gas turbine compartment and a gas turbine, wherein the controller enables continued operation of the gas turbine in response to determining that the selected sensor is operating after the recalibrating.

15. The gas detection system of claim 12, wherein the predetermined gas includes a hazardous gas.

16. The gas detection system of claim 12, further comprising a source of negative pressure connected to each of the first sensing location and the second sensing location to direct gas to each of the first gas sensor and the second gas sensor.

17. The gas detection system of claim 16, wherein the source of negative pressure includes a fan.

18. The gas detection system of claim 12, wherein the valve system includes at least two solenoid three-way valves.

19. The gas detection system of claim 12, wherein the valve system includes at least two solenoid two-way valves.

20. The gas detection system of claim 12, wherein the first sensing location and the second sensing location are each connected to the respective gas sensor by at least one sampling conduit and the valve system.
